(19) United States
(12) Patent Application Publication
(10) Pub. No.: US 2008/0249374 A1
(43) Pub. Date: Oct. 9, 2008

(54) METHOD AND APPARATUS TO ENABLE MULTIPLE METHODS OF CLINICAL ORDER INPUT INTO A HEALTHCARE IT APPLICATION

(75) Inventors: Mark M. Morita, Arlington Heights, IL (US); William Douglas Hughes, Bainbridge Island, WA (US)

Correspondence Address: MCANDREWS HELD & MALLOY, LTD, 500 WEST MADISON STREET, SUITE 3400, CHICAGO, IL, 60661, US

(73) Assignee: General Electric Company, Schenectady, NY (US)

(21) Appl. No.: 11/697,384

(22) Filed: Apr. 6, 2007

(51) Int. Cl. A61B 5/00

(52) U.S. Cl. 600/300

(57) ABSTRACT

The present invention relates to a system for facilitating the input of clinical orders into a healthcare application. This system can provide a variety of ways for a user to interface with the system and a variety of ways to facilitate data input into an order form. The system can facilitate selection of an order form by selecting an order form for the user, or by providing the user with a list of order forms that the user can select an order form from. The system facilitates the input of data into an order form in several ways. This system can use stored information to pre-populate some of the information fields in an order form. Data input can also be facilitated by the use of predictive typing, keywords, and drop-down lists. The system can also facilitate data input by taking free form text, parsing the text and entering the text into at least one information fields on an order form.

### FIELD OF THE INVENTION

The present invention relates to a system for facilitating the input of clinical orders into a healthcare application. More particularly, this system can provide a variety of ways for a user to interface with the system, and variety of ways to facilitate data entry into a clinical order form.

### BACKGROUND OF THE INVENTION

Currently, when placing clinical orders many doctors take notes on paper and have assistants enter these orders at a later time. Taking notes on paper and then having assistants enter these orders is inefficient because it requires assistants to spend extra time inputting orders, and delays the entry of orders until an assistant is able enter the orders. Furthermore, reading the handwriting in a Doctor's note can sometimes be difficult for assistants, increasing the chance that an assistant will incorrectly enter a clinical order.

The healthcare software applications that currently exist for clinical order entry do not allow healthcare workers to easily input clinical orders. Currently existing software often utilizes traditional hierarchical menus trees with multiple pull down menus, making the use of the software difficult and time consuming. Furthermore, this software often requires a user to re-enter all of the order and patient information each time an order is placed, instead of allowing the system to store and access previously entered information.

Healthcare providers are typically mobile, and have to move from room to room to see different patients. As healthcare providers are typically mobile, and typically have a limited amount of time to spend with each patient, it would be beneficial if there was a mobile system that provided for a simpler and easier method of entering clinical orders. It would also be beneficial if order information, patient information, and other medical information could be stored by a system and later selected by a user so that clinical orders could be entered more easily.

### SUMMARY OF THE INVENTION

Accordingly, certain embodiments of the present invention provide systems and methods which facilitate clinical order entry. The system can facilitate order entry by providing keywords, drop-down lists, and predictive typing. The system can also facilitate data entry by taking free form text, formatting the free form text as standard data and entering it into at least one information field.

Certain embodiments of the present invention provide a system for using stored information to pre-populate information fields. The stored information used to pre-populate an order form can be, but is not limited to, patient history information, patient charts, notes, stored clinical orders or information stored in the system about medications, and clinical tests.

Certain embodiments of the present invention facilitate selection of an order form. Order form selection can be facilitated by enabling the system to select an order form for the user. The system can also facilitate order form selection by providing a drop-down list of order forms to the user, and enabling the user to select a form from the list. The system can create this drop-down list of order forms using stored data, data input by the user, and/or data selected by the user from another drop-down list. In this way the system can provide a user with a list of forms that is related to the clinical order being placed by the user.

Another embodiment is a method of entering clinical orders by receiving input data from a user interface, facilitating selection of an order form using the input data and stored data, where the entry of data into information fields is facilitated, where the facilitated entry of data into the information fields uses at least one of keywords, predictive typing, drop-down lists, and pre-populating the information fields, and where selection of the order form is facilitated by at least one of selecting the order form for a user and providing a list of order forms for a user to select an order form from.

Certain embodiments provide computer-readable storage medium including a set of instructions for a computer, where the set of instructions has a user interface routine for receiving input data, an order form selection routine capable of facilitating selection of the order form using the input data and stored data, an information field entry routine using at least one of keywords, predictive typing, drop-down lists, and pre-populating to facilitate entry of information into the information field, where the order form selection routine comprises at least one of selecting an order form for a user and providing a drop-down list of order forms for a user to select from.

Another embodiment provides a computer-readable storage medium including a set of instructions for a computer, where there is a drop-down list routine for forming the drop-down list from stored data, input data, the category of the information field, and data in other information fields, and where a user can select order information from the drop-down list in order to fill out the information field.

Certain embodiments provide a computer-readable medium having computer-executable instructions for performing a method wherein there is a free form text routine, which converts free form text to standard format data, and where the standard format data is entered into at least one information field.

Certain embodiments provide a computer-readable storage medium including a set of instructions for a computer, where the user interface routine can receive input data from at least one of a keyboard, mouse, touchpad, and voice interface.

These and other features of the present invention are discussed or apparent in the following detailed description.

### BRIEF DESCRIPTION OF THE DRAWINGS

FIG. 1 is an illustration of a system for clinical order entry, in accordance with an embodiment of the present invention.

FIG. 2 is an illustration of a user interface for the entry of data for clinical orders into a healthcare IT application in accordance with an embodiment of the present invention. The entry of input data can trigger a drop-down list and data can be selected from the drop-down list.

FIG. 3 is an illustration of an order form that has been selected for clinical order entry into a healthcare IT application.

FIG. 4 is an illustration of a user interface for the entry of data for clinical orders into a healthcare IT application.

FIG. 5 is an illustration of a flow diagram for a method of facilitating the entry of clinical orders.

The foregoing summary, as well as the following detailed description of certain embodiments of the present invention, will be better understood when read in conjunction with the appended drawings. For the purpose of illustrating the invention, there is shown in the drawings, certain embodiments. It should be understood, however, that the present invention is not limited to the arrangements and instrumentality shown in the attached drawings.

### DETAILED DESCRIPTION

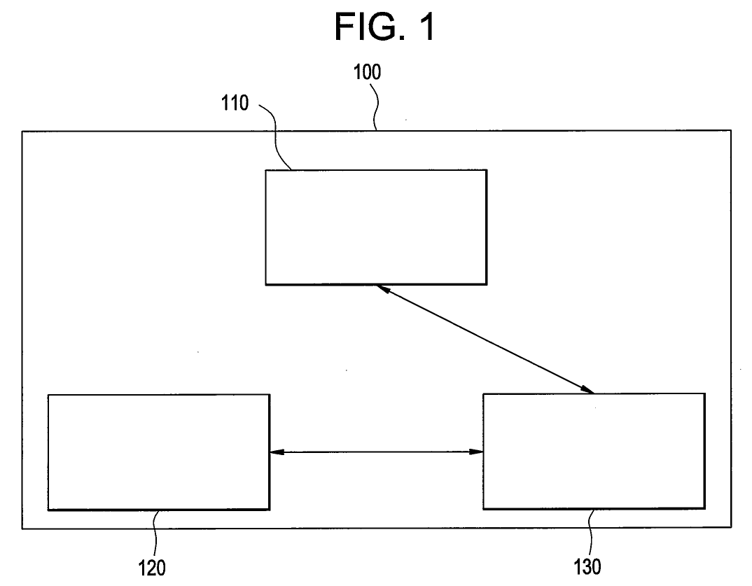

In accordance with certain embodiments, FIG. 1 illustrates a system 100 for clinical for order entry. The system for clinical order entry includes a user interface 110, memory 120, and a processor 130. The memory 120 of the system 100 can contain stored data. The stored data can contain, but is not limited to, clinical order information about medicines and medical tests that can be ordered by a user. For each drug that can be ordered, the stored clinical order information can include the diagnoses that each drug can be used to treat, doses, formulations, route of delivery, frequency, duration, refills, and further instructions. The stored data can also contain stored patient information. Stored patient information can include, but is not limited to, the medical history of a patient, and previous clinical orders for the patient. The medical history of the patient can include information such as past medical conditions, and background information such as the age and gender of the patient. Data can be input into the system by a user through the user interface 110. The processor 130 of the system can use input data and stored data to facilitate selection of an order form, and provide facilitated entry of data into the information fields of the order form.

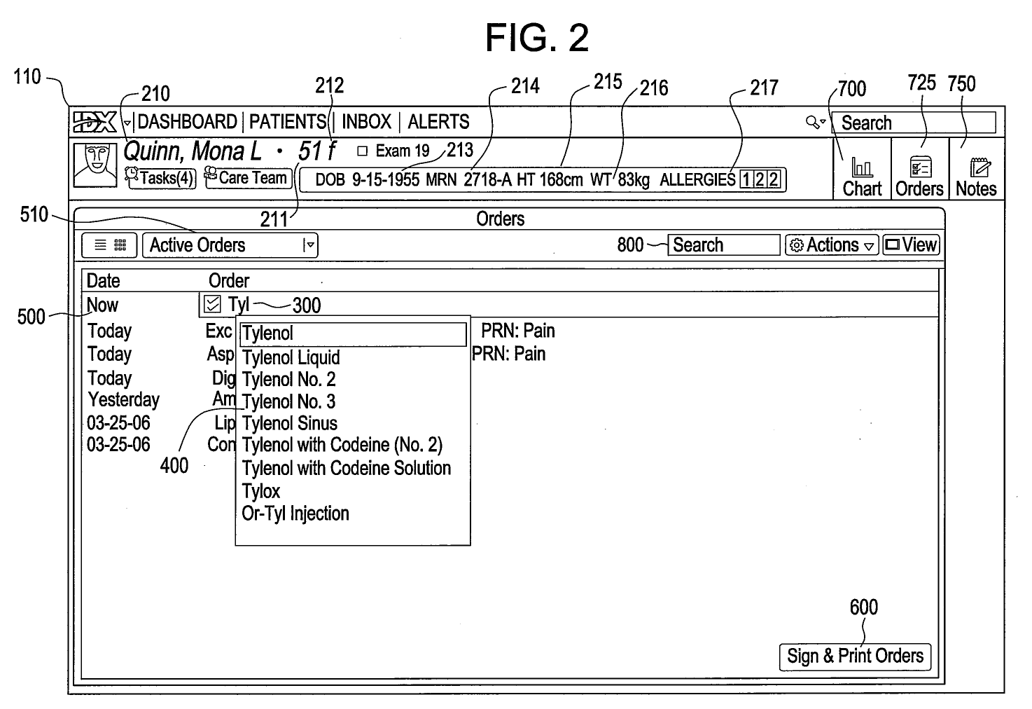

In one embodiment, shown in FIG. 2, the user interface can be provided for clinical order entry. The user interface 110 in FIG. 2 has information fields, which can include patient name 210, patient age 211, patient gender 212, Date of birth (“DOB”) 213, Medical record number (“MRN”) 214, height (“HT”) 215, weight (“WT”) 216, and “allergies” 217. The information in the user interface information fields can be entered and/or pre-populated with patient information before the order form 200 has been selected. In another embodiment, the information in the user interface information fields can be entered and/or pre-populated after the order form has been selected.

In an embodiment, shown in FIG. 2, the user interface 110 also has a text entry box 300. Text can be entered into the text entry box 300 by any of a plurality of devices including keyboard, mouse, touchpad, and voice interface. When the user begins entering information into the text entry box 300, the system can provide a drop-down list 400, for example. The drop-down list 400 can allow a user to select information, thereby facilitating order entry. However, if a user wants to input information into an information field that is not provided in the drop-down list 400, the user does not have to select information from the drop-down list 400. Instead, the user can enter information into an information field.

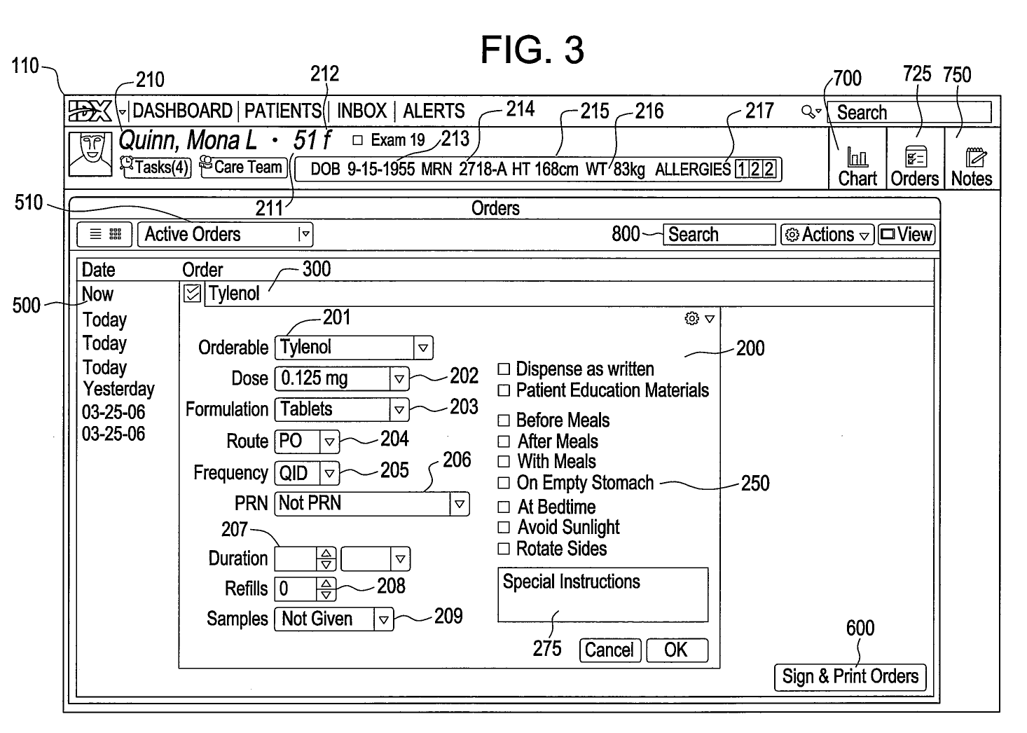

FIG. 3 shows a user interface 110 an order form 200 which has a text entry box 300. The system can facilitate the selection of an order form 200 by using user input data and/or information selected by the user from a drop-down list 400. The system can then select an order form for the user or provide the user with a drop-down list 400 of order forms from which the user can select an order form.

In FIG. 3 the order form 200 has information fields. The information fields in an order form 200 can include, but are not limited to, “Orderable” 201, “Dose” 202, “Formulation” 203, “Route” 204, “Frequency” 205, “PRN” 206, “Duration” 207, “Refills” 208, and “Samples” 209. The order form 200 can also have at least one information field for insurance information.

The information fields can have categories associated with the information field. The category of the information field can describe the type of information in the information field. The category of the information field can be displayed on the order form 200, however, the category does not have to be displayed on the order form 200. For example, in the embodiment shown in FIG. 3 the field 201 has the label “Orderable” next to field 201 indicating that the category of the field is for the orderable and that an orderable can be selected or entered into this information field. However, in FIG. 3 the patient gender field 212 displays “f.”

The order form can also include further instructions 250, where boxes containing further instructions can be checked if they apply. These boxes can be checked by the user or they can be filled out by the system, using stored information about the orderable and/or patient information. In one embodiment the further instructions 250 can include instructions for taking medication, such as “Before Meals,” “After meals,” “With Meals,” “On Empty stomach,” “At bedtime,” or “rotate sides.” In the embodiment shown in FIG. 3 the further instructions 250 displayed on the order form 200 can be different on different order forms 200. In the embodiment shown in FIG. 3, the further instructions 250 can also include directions such as providing “Patient Education Materials” with the medication, “Dispense as written” or for a patient to “Avoid sunlight” when taking the medication. In an embodiment where an order form 200 is used to order a test, the further instructions 250 can include boxes that can be checked to indicate the restrictions on food or water that the patient needs to follow before the test. The order form 200 can also include a space where a user can enter Special Instructions 275. The Special Instructions 275 allows a user to customize the form to include instructions or notes that are not included in the further instruction section 250 on the order form 200 which has been selected. The user can also create additional information fields on an order form 200 to further customize the order form 200.

The present system provides facilitated clinical order entry. This system can use stored patient information to pre-populate some of the information fields. For example, if a patient name 210 has been input or selected, the system can fill out the information field for the Medical Reference Number 214. Also if the MRN has been input or selected in the MRN field 214 the system can fill out the patient name field 210. Furthermore, the system can also pre-populate information fields by filling out the information fields for the patient's age 211, the patient gender 212, Date of birth (“DOB”) 213, height (“HT”) 215, allergies 217 and other information from the patient's medical history. Information fields on an order form can also be pre-populated. Pre-populating the order form saves the user time, since the user does not have to re-enter information that is stored or information that the system can access. However, if any of the information in the pre-populated information fields needs to be changed it is possible for the user to change the information in those fields.

Stored patient information and other stored information can be used to provide a drop-down list 400 in each information field. The drop-down list 400, allows a user to quickly select information to fill out an information field. In one embodiment, the drop-down list 400 can contain a list of medications or tests ordered for the patient in the past, enabling a user to quickly order a medication or a test that was previously ordered. For example, if the patient was previously prescribed Nexium® and Lipitor®, “Nexium®” and “Lipotor®” could be provided in the drop-down list 400 in the “Orderable” field 201. Also, the stored information can be used by the system to provide a drop-down list 400 of medications or tests that can be ordered for a particular diagnosis. For example, if a diagnosis field indicates that the patient has heartburn the drop-down list 400 can contain a list of medicines that are prescribed for heartburn, such as Nexium®. The user can then easily select a medicine from the choices listed in the drop-down list 400, without needing to input the full medication name into the “Orderable” information field 201.

In another embodiment up and down arrows can be used to facilitate entry of information into information fields. The up and down arrows can be provided for an information field that does not provide a drop-down list. The up and down arrows can also be provided for an information field, which has a drop-down list. In the embodiment illustrated in FIG. 3 the “Duration” information field 207 provides up and down arrows to facilitate entry of information. The up and down arrows can be provided along with drop-down lists, as illustrated in the “Duration” field 207 of FIG. 3. The up arrow can be used to increase the information in the information field, and the down arrow can be used to decrease the information in the information field. For example, in FIG. 3 the “Refills” field 208 has “0” entered in the field. To increase the number in the “Refills” field 208 from “0” to “3” the up arrow could be used. The up and down arrows can also be provided to facilitate information entry when a drop down list is not provided, as shown in the “Refills” field 208 of FIG. 3. In another embodiment, the user can select the number of refills for the “Refills” field 208 from a drop-down list.

In another embodiment of the present system, the drop-down list 400 for an information field can contain clinical order information that is used in common clinical orders. In another embodiment the drop-down list 400 can contain clinical order information that is used in common clinical orders, but does not display clinical orders that are not commonly ordered.

The system can also facilitate order entry by providing predictive typing. With predictive typing, letters entered by the user into the text entry box 300 can prompt the system to provide information that begins with those letters in a drop-down list 400. For example, entering the letter “C” then the letter “L” could prompt the system to provide the names of the drugs “Cleocin®” and “Clarinex®.” However, in an embodiment the choices suggested by the predictive typing could also be focused based on input data and stored clinical order information. For example, if the order form indicates that the diagnosis is related to allergies, the predictive typing would not suggest the acne medicine Cleocin®. Instead Clarinex® might be one of the drugs prompted by the predictive typing, because the stored clinical order information can indicate that Clarinex® can be used in the treatment of allergies.

The system can also facilitate order entry by the use of keywords. Once a keyword is inputted into the system this can provide the user with a drop-down list 400 containing information related to the keyword.

In FIG. 2 data can be entered in the text entry box 300. For example, in the embodiment of the system shown in FIG. 2 the user can enter “Ty” into the text entry box 300, and the system can use predictive typing to provide a drop-down list 400. The drop-down list 400 includes Tylenol®, Tylenol® Liquid, Tylenol® No. 2, Tylenol® No. 3, Tylenol® Sinus, Tylenol® with Codeine (No. 2), Tylenol® with Codeine Solution, Tylox®, and Or-Tyl® Injection, for example. The user can then select one of the choices from the drop-down list 400. Once the user has selected a choice from the drop-down list 400 this keyword can be used to facilitate selection of an order form.

In the embodiment shown in FIG. 3 the patient name 210 and/or MRN 214 can also be used to access stored patient information and previous orders for the patient. The accessed information can then be used to pre-populate the user interface information fields and the order form information fields. For example, in FIG. 3 the user interface information fields for “Quinn, Mona L” could be pre-populated by the system so that the patient age field 211 indicates that the patient is “51,” patient gender field 212 states “f,” the Date of birth field 213 states “9-15-1955,” the MRN field 214 states “2718-A,” and that allergy information is entered into the allergies field 217. The information in the user interface information fields can be entered and/or pre-populated with patient information before the order form 200 has been selected. In another embodiment, the information in the user interface information fields can entered and/or pre-populated after the order form has been selected. The information fields in the user interface and the order form can be pre-populated with information from, but not limited to, the patient's medical history.

In the embodiment shown in FIG. 3 the selected order form 200 can provide information fields related to ordering the keyword, “Tylenol.” These order form information fields include “Orderable” 201, “Dose” 202, “Formulation” 203, “Route” 204, “Frequency” 205, “PRN” 206, “Duration” 207, “Refills” 208, and “Samples” 209. In the present embodiment the system can then enter the keyword Tylenol® into the “orderable” field 201. The user can then input or select information in each of the order form information fields. To facilitate order entry the system can provide a drop-down list 400 for each order form information field. For example, the system can provide common dosages of Tylenol® in a drop-down list 400 for the “Dose” field 202. The order form information fields can be pre-populated with selected information or input data. For example in FIG. 3, the “Orderable” information field can be pre-populated with “Tylenol.”

In another example, the user can input “diabetes” into the text entry box 300. The system can then provide a drop-down list 400 of order forms related to diabetes or the system can select a form for the user based on, but not limited to, stored data, input data or data selected from a drop-down list 400. The list of forms in the present example can include a clinical order form 200 for diabetes medication, or a test related to diabetes. These order forms 200 could then have information fields that allow for the entry of the “A1C scores,” “blood glucose scores,” or “blood pressure information.” If information relating to these tests was previously stored, the user can be provided a drop-down list 400 of test results that the user can select from in order to enter information in the information fields.

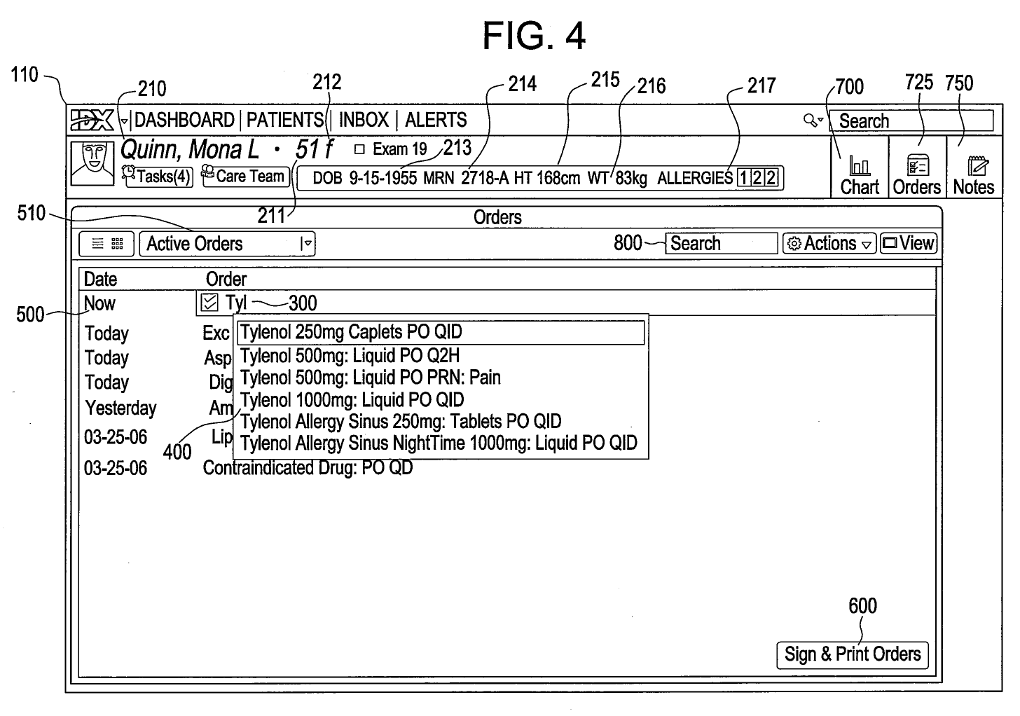

In one embodiment, input that has been entered into the text entry box 300 as free form text and/or free form text that has been selected from a drop-down list 400 can then be parsed and converted into standard format for a clinical order. The standard format data can then be entered into at least one information field on the order form. For example, in the embodiment shown in FIG. 4 the user can select the free form text stating “Tylenol 250 mg Caplets PO QID” from a drop-down list 400. The system can then parse the free form text, convert the data to a standard format and the standard format data into at least one information field on the order form. Therefore, in the present example “Tylenol” would be entered into the “Orderable” field 201, “250 mg” would be entered into the “Dose” field 202, “Caplets” would be entered in the “Formulation” field 203, “PO” would be entered in the “Route” field 204, and “QID” would be entered in the “Frequency” field 205. This allows for facilitated order entry into a clinical order form.

In another embodiment, information copied or dragged by a user from other applications, order forms, programs, systems, and other parts of the IT software, can be converted to standard format data. The standard format data for clinical order entry can then be entered into information fields in the order form 200. Information that is copied or dragged can include patient charts, and notes. Other order forms can also be copied or dragged and then be utilized. Copying or dragging information facilitates the entry of information into the order form 200, and can also facilitate the entry of clinical orders that are not commonly used.

This system can provide the additional safety of checking to determine if an orderable is known to cause an adverse drug reaction when taken with other medications the patient is taking. If the system determines that an order could cause a potential adverse reaction, the system will provide a warning to the user. The system can also use stored information about a patient's known allergies, to provide a warning to the user if patient is known to be allergic to an orderable. For example, if the orderable is “Penicillin” and it is known that the patient is allergic to Penicillin a warning will be provided to the user. In another embodiment, the user will not be able to enter the orderable and/or complete the clinical order if it is known that the patient is allergic to the orderable.

This system also allows users to input order information using a variety of user interfaces. These user interfaces can include a keyboard, mouse, touchpad, and voice interface. The system can be used in conjunction with a variety of devices including a word processor, keyboard, tablet PC, voice dictation system, PDA, Smartphone, Bluetooth® headset, palm pilot, and Blackberry®, for example.

The user can access previous orders from the “Order” button 725, the order history 500, and “active orders” drop-down menu 510. The user can also access stored patient information from the “Chart” button 700, and “Notes” button 750. The information from the patient charts 700 and notes 750 can be viewed by the user, copied and/or dragged into the order form 200. The user can also search stored data using the search box 800.

Once an order form 200 has been completed the user can click on the “Sign & Print Orders” button. The order can be saved, and can be accessed by the user using the order history 500, the “Order” button 725, the “Active Order” drop-down menu 510, and/or the search box 800.

Certain embodiments provide a method of facilitating input of data into information fields using at least one of keywords, predictive typing, drop-down lists 400, and pre-populating the information fields.

Certain embodiments provide a method where a drop-down list for an information field is formed using the category of the information field, data in other information fields, stored data, input data, and the order form. The user can then select data from the drop-down list, and the selected data will be entered into the information field.

Certain embodiments provide a method where free form text is input; the free form text is parsed; the parsed free form text is converted to standard format data; and the standard format data is input into at least one of the information fields.

Certain embodiments provide a method of entering clinical orders where the user interface can be, but is not limited to, a keyboard, mouse, touchpad, and voice interface.

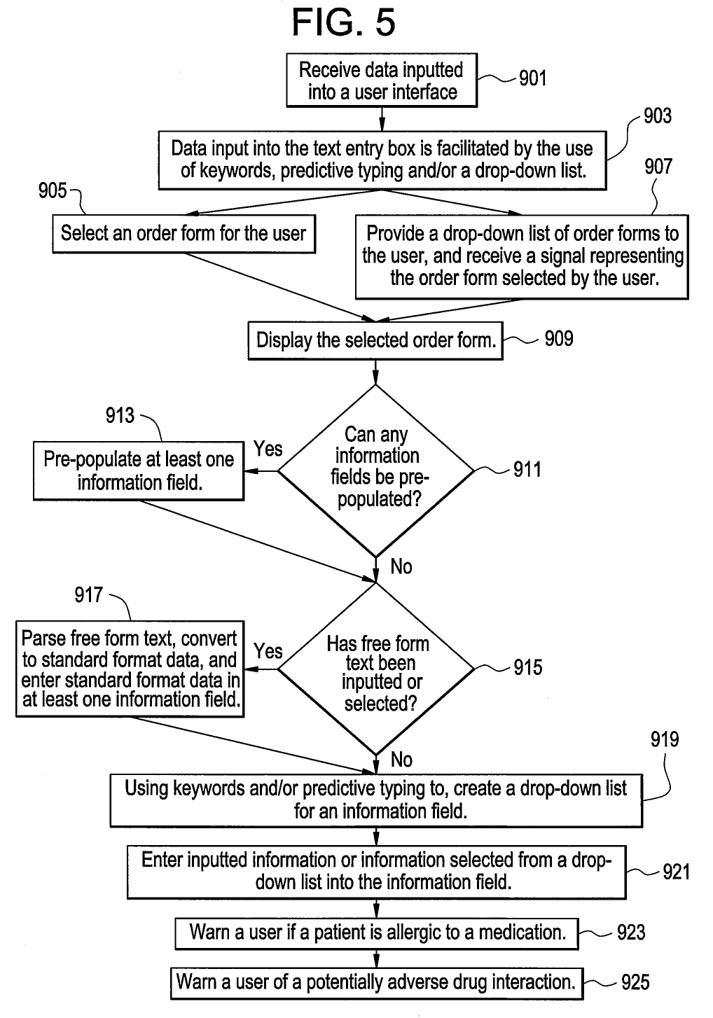

FIG. 5 illustrates a flow diagram for a method of facilitating the entry of clinical orders in accordance with an embodiment of the present invention. The method includes the following steps. First, at step 901, input data is received from a user interface. Next, at step 903, data input into the text entry box 300 can be facilitated by at least one of using keywords, predictive typing and/or a drop-down list. Next, step 905 or step 907 can occur. At step 905, an order form is selected for the user. At step 907, a drop-down list of order forms is provided to the user and a signal representing the order form selected by the user is received. At step 909, the selected order form is displayed. At step 911, it is determined whether any information fields can be pre-populated. If no information fields are going to be pre-populated then step 915 occurs. If any information fields can be pre-populated, those information fields are pre-populated at step 913, and then step 915 occurs. At step 915, it is determined whether any free form text has been inputted or selected. If free form text has not been inputted or selected by a user then step 919 occurs. If free form text has been inputted step 917 occurs. At step 917, free form text is parsed, converted to standard format data and the standard format data is entered into at least one information field. At step 919, data entry into an information field is facilitated by the use of keywords and/or predictive typing to create a drop-down list for the information field. Next, at step 921, information is entered into the information field. This information can be information selected from a drop-down list or information inputted into the information field by the user. Steps 919 and 921 can be repeated for each information field in the order form 200. At step 919, if there is no drop-down list for an information field, step 921 occurs and the user can input information. The user can also input information even if a drop-down list has been created. In one embodiment, the input of information can be facilitated by the use of up and down arrows. At step 923, a user can be warned if a patient has a known allergy to the orderable 201. At step 925, a user can be warned if a potentially adverse drug reaction can occur between medications. The potentially adverse drug reaction can occur between drugs that are being ordered, or between drugs being ordered for a patient and drugs the patient is already taking.

For example, at step 901 the user interface could receive the input data “Ty.” Next, at step 903, data input into the text entry box 300 data can be facilitated by the use of predictive typing to provide a drop-down list. For example, the drop-down list 400 can include Tylenol®, Tylenol® Liquid, Tylenol® No. 2, Tylenol® No. 3, Tylenol® Sinus, Tylenol® with Codeine (No. 2), Tylenol® with Codeine Solution, Tylox®, and Or-Tyl® Injection. The user can then select Tylenol® from the drop-down list. Next, at step 905, an order form for ordering Tylenol® can be selected for the user. At step 909, the selected order form for ordering Tylenol® is displayed. At step 911, it is determined that an information field can be pre-populated. At step 913, the “orderable” field 201 is pre-populated with “Tylenol®.” At step 915, it is determined that no free form text has been inputted, therefore, next step 919 occurs. At step 919, a drop-down list 400 is created for the “formulation” field 203. For example, the drop-down list can include “Caplets,” “Tablets” and “Liquid.” The user can then select “Tablets” from the drop-down list. At step 921, “Tablets” would be entered into the “formulation” field 203. Steps 919 and 921 can be repeated, for each information field. For example, a drop down list can be created for the “Frequency” field 205. The drop down list for the “Frequency” field 205 could include “Q2H,” “QQH,” “QID,” and “QD.” The user can then select “QID” from the drop-down list. At step 919, if there is no drop-down list for an information field, step 921 occurs and the user can input information. The input of information can be facilitated by the use of up and down arrows, as illustrated in FIG. 3. For example, in FIG. 3 the “Refills” field 208 has up and down arrows, which can be used to increase the number of refills from “0” to “2.” At step 923, a warning can be provided if the patient is known to be allergic to the orderable. For example, if the orderable is “Tylenol,” and it is known that the patient is allergic to Tylenol, at step 923 a warning will be provided to the user. In another embodiment, the user will not be able to enter the orderable and/or complete the clinical order if it is known that the patient is allergic to the orderable. At step 925, a user can be warned of a potentially adverse drug reaction. For example, if a patient is taking a drug that can cause an adverse reaction with Tylenol, a warning can be provided to the user.

Certain embodiments of the present invention may omit one or more of these steps and/or perform the steps in a different order than the order listed, including simultaneously.

Certain embodiments provide computer-readable storage medium including a set of instructions for a computer, the set of instructions having a user interface routine for receiving input data, an order form selection routine capable of facilitating selection of the order form using the input data and stored data, an information field entry routine using at least one of keywords, predictive typing, drop-down lists, and pre-populating, to facilitate entry of information into the information field, where the order form selection routine at least one of selects an order form for a user and provides a drop-down list of order forms for a user to select from.

Certain embodiments provide a computer-readable storage medium including a set of instructions for a computer, where there is a drop-down list routine for forming the drop-down list from at least one of stored data, input data, the category of the information field, and data in other information fields, and where a user can select order information from the drop-down list in order to fill out the information field.

Certain embodiments provide a computer-readable medium having computer-executable instructions for performing a method where there is a free form text routine, which converts free form text to standard format data, and where the standard format data is entered into at least one information field.

Certain embodiments provide a computer-readable storage medium including a set of instructions for a computer, where the user interface routine can receive input data from at least one of a keyboard, mouse, touchpad, and voice interface.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A system for clinical order entry comprising:
a user interface accepting input data from a user;
a memory comprising stored data; and
a processor using said input data and said stored data to facilitate selection of an order form, wherein said order form has at least one information field, and wherein said processor provides facilitated entry of data into at least one of said information fields.

2. The system for clinical order entry of claim 1 wherein said processor facilitates selection of said order form using said input data and said stored data by at least one of selecting an order form for said user and providing said user with a list of order forms, wherein said user can select an order form from said list of order forms.

3. The system for clinical order entry of claim 1 wherein said facilitated entry of data into at least one of said information fields comprises at least one of predictive typing, the use of keywords and providing a drop-down list for said information fields.

4. The system for clinical order entry of claim 3 wherein said information field has a category, and wherein at least one of said category of said information field, said order form, said stored data, said input data, and data in other of said information fields is used to form said drop-down list, and wherein said user can select order information from said drop-down list in order to enter data into at least one of said information fields.

5. The system for clinical order entry of claim 4 wherein said category of said information field comprises at least one of patient name, age, date of birth, medical reference number, height, weight, allergies, diagnosis, orderable, dose, formulation, route, frequency, PRN, duration, refills, samples, procedure, test result, test order, and insurance information.

6. The system for clinical order entry of claim 1 wherein said stored data used to form said drop-down list comprises at least one of previous orders for a patient, previous patient information, and clinical order information, wherein said clinical order information comprises orderable test information, and orderable drug information, wherein said orderable drug information comprises at least one of the orderable drug, doses, formulations, routes of delivery, frequency, duration, refills, and further instructions for each diagnosis.

7. The system for clinical order entry of claim 4 wherein said drop-down list comprises only said clinical order information that is commonly ordered.

8. The system for clinical order entry of claim 1 wherein said user can customize said order form by at least one of adding additional said information fields, selecting further instructions, and adding special instructions.

9. The system for clinical order entry of claim 1 wherein said user interface comprises at least one of a keyboard, mouse, touchpad, and voice interface.

10. The system for clinical order entry of claim 1 wherein said system can identify a possible adverse drug interaction, and wherein said system can warn said user of an allergy to a clinical order.

11. The system for clinical order entry of claim 1 wherein said input data comprises free form text, and wherein said system converts said free form text to standard format data, and said standard format data is entered into at least one of said information fields.

12. The system for clinical order entry of claim 1 wherein there is information outside of said order form, wherein said information outside of said order form can be at least one of copied and dragged into said order form, and wherein said information outside of said order form comprises at least one of another order form in said system, patient charts, notes, information from another system, and information from another application.

13. A method of entering clinical orders comprising:
receiving input data from a user interface;
facilitating selection of an order form using said input data and stored data to at least one of select said order form for a user and provide a list of order forms for a user to select from; and
facilitating entry of data into information fields using at least one of keywords, predictive typing, drop-down lists, and pre-populating said information fields.

14. The method of entering clinical orders of claim 13 wherein said drop-down list is formed from said at least one of said stored data, said input data, said order form, the category of a first of said information fields, and data in other of said information fields, and wherein said user can select data from said drop-down list and wherein said user selected data is entered into said first information field.

15. The method of entering clinical orders of claim 13 wherein said input data comprises free form text, and wherein said free form text is parsed, wherein said parsed free form text is converted to standard format data, and wherein said standard format data is entered into at least one of said information fields.

16. The method of entering clinical orders of claim 13 wherein said user interface comprises at least one of a keyboard, mouse, touchpad, and voice interface.

17. A computer-readable storage medium including a set of instructions for a computer, the set of instructions comprising:
a user interface routine for receiving input data;
an order form selection routine capable of facilitating selection of said order form using said input data and stored data to at least one of select an order form for a user and provide a drop-down list of order forms for a user to select from; and
an information field entry routine using at least one of keywords, predictive typing, drop-down lists, and pre-populating, to facilitate entry of information into said information field.

18. The computer-readable storage medium including a set of instructions for a computer, of claim 17 wherein there is a drop-down list routine for forming a drop-down list from at least one of said stored data, said input data, the category of said information field, and data in other of said information fields, and wherein a user can select order information from said drop-down list in order to fill out said information field.

19. The computer-readable storage medium including a set of instructions for a computer, of claim 17 wherein there is a free form text routine, wherein free form text is converted to standard format data, and wherein said standard format data is entered into at least one of said information fields.

20. The computer-readable storage medium including a set of instructions for a computer, of claim 17 wherein said user interface routine can receive input data from at least one of a keyboard, mouse, touchpad, and voice interface.
